(19) United States
(12) Patent Application Publication
(10) Pub. No.: US 2006/0276463 A1
(43) Pub. Date: Dec. 7, 2006

(54) Pure levofloxacin hemihydrate and processes for preparation thereof

(76) Inventors: Tarun Kant Sharma, Bihar (IN); Pramed Kumar, Uttar Pradesh (IN); Prosenjit Bose, Gurgaon, Haryana (IN); Yatendra Kumar, Gurgaon, Haryana (IN)

Correspondence Address: RANBAXY INC., 600 COLLEGE ROAD EAST, SUITE 2100, PRINCETON, NJ, 08540, US

(21) Appl. No.: 10/539,130

(22) PCT Filed: Dec. 15, 2003
(86) PCT No.: PCT/IB03/05945

§ 371 Date: Jun. 19, 2006

(30) Foreign Application Priority Data
Dec. 16, 2002 (IN) 1263/DEL/2002

(51) Int. Cl. A61K 31/538; C07D 491/04

(52) U.S. Cl. 514/229.8; 544/101

(57) ABSTRACT

The invention relates to pure levofloxacin hemihydrate and a process for preparing pure levofloxacin hemihydrate. The invention also relates to pharmaceutical compositions that include the pure levofloxacin hemihydrate and use of said compositions for treating a patient in need of an antimicrobial therapy.

### FIELD OF THE INVENTION

The field of the invention relates to pure levofloxacin hemihydrate and a process for preparing pure levofloxacin hemihydrate. The invention also relates to pharmaceutical compositions that include the pure levofloxacin hemihydrate and use of said compositions for treating a patient in need of an antimicrobial therapy.

### BACKGROUND OF THE INVENTION

Chemically, levofloxacin is hemihydrate crystals of (S)-9-fluoro-3-methyl-10-(4-methyl-1-piperazinyl)-7-oxo-2,3-dihydro-7H-pyrido[1,2,3de][1,4]benzoxazine-6-carboxylic acid having structural Formula I, which is used for treating bacterial infections.

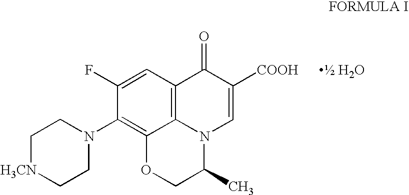

It is a well-known antimicrobial agent, and is active against a broad spectrum of gram-positive and gram-negative bacteria. Levofloxacin is particularly more effective against Streptococcus and Staphylococcus strains of bacteria.

Levofloxacin exists as hemihydrate crystalline form of structural Formula I, and monohydrate form of structural Formula II. It also exists as anhydrous crystals which can be obtained by dehydrating the hemihydrate and monohydrate forms.

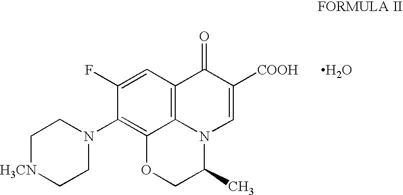

U.S. Pat. No. 5,053,407 discloses a process for the preparation of levofloxacin, which involves recrystallization of levofloxacin from a solvent mixture of ethanol and diethyl ether. The use of a solvent mixture results in the production of levofloxacin monohydrate, together with the desired levofloxacin hemihydrate form.

There are significant drawbacks to this approach as the conversion of the monohydrate into the hemihydrate is difficult to achieve and requires reprocessing using repeated crystallizations. The crystallization requires large quantities of solvents and results in significant loss of product. Alternatively, when an attempt is made to remove the crystal water from the monohydrate by drying, it results in the formation of anhydrous crystals. When the anhydrous crystals thus obtained are allowed to take up moisture to get the desired hemihydrate, it only results in the formation of the monohydrate. Furthermore, anhydrous crystals obtained by removing crystal water are very difficult to isolate as they have a tendency to stick to the walls of the vessel or reactor.

U.S. Pat. No. 5,545,737 discloses a process for the preparation of levofloxacin hemihydrate or monohydrate wherein crude crystals of levofloxacin are dissolved in aqueous solvent system selected from methanol, ethanol, propanol and acetone having a specific percentage of water, the solution is treated with activated carbon and after filtration is concentrated to a specific volume and then cooled and filtered to obtain hemihydrate.

The prior art approach is not suitable from commercial point of view because the desired product is not obtained in high purity and is more time consuming, thus making the approach commercially difficult to implement. The purity hereto refers to the compound purity, enantiomeric purity as well as purity with respect to the hydrated form.

To achieve a high efficiency of reaction for industrial scale synthesis of levofloxacin hemihydrate, it is necessary to minimize the formation of the monohydrate along with levofloxacin hemihydrate.

Thus, the present invention provides a process which does not result in a mixture of hydrated forms; rather pure form is obtained which does not require further purification. The choice of solvents has been found to be important for obtaining the pure product.

### SUMMARY OF THE INVENTION

In one general aspect there is provided a pure levofloxacin hemihydrate.

The pure levofloxacin hemihydrate may have the X-ray diffraction pattern of FIG. 1.

In another general aspect there is provided pure levofloxacin hemihydrate crystals, which are essentially free from levofloxacin monohydrate form.

In another general aspect there is provided a pharmaceutical composition that includes a therapeutically effective amount of pure levofloxacin hemihydrate crystals; and one or more pharmaceutically acceptable carriers, excipients or diluents.

In another general aspect there is provided a process for the preparation of pure levofloxacin hemihydrate crystals. The process includes obtaining a solution of crude levofloxacin in one or more organic solvents; removing the solvent; maintaining a moisture content of reaction mass from about 0.5% w/w to about 1.5% w/w; and isolating the pure levofloxacin hemihydrate.

The solvent may be one or more of chlorinated hydrocarbon, hydrocarbon, ester, water, or mixtures thereof. The chlorinated hydrocarbon may include one or more of chloroform, dichloromethane, and 1,2-dichloroethane. In particular, the chlorinated hydrocarbon includes dichloromethane.

The hydrocarbon may include one or more of hexanes, cyclohexane, and toluene. The ester may include one or more of methyl acetate, ethyl acetate, and isopropyl acetate. In particular, the ester includes ethyl acetate. The solvent may be removed by a technique which includes one or more of distillation and distillation under vacuum. Isolating the pure levofloxacin hemihydrate includes one or more of filtration, filtration under vacuum, decantation and centrifugation.

The process may include further drying of the product obtained.

The process may produce the pure levofloxacin hemihydrate having the X-ray diffraction pattern of FIG. 1 and the moisture content of from between about 2.4% to about 2.5%.

In one general aspect, the solution of crude levofloxacin may be obtained by heating the solvent containing crude levofloxacin. It may be heated from about 30° C. to about reflux temperature of the solvent used, for example from about 30° C. to about 100° C. In particular, it may be heated from about 40° C. to about 60° C. It may be heated from about 15 minutes to about 10 hours. More particularly, it may be heated for about 2-3 hours.

In another general aspect, a base may be added prior to heating the solvent containing crude levofloxacin. Any base may be used, for example triethylamine.

The moisture content of the reaction mass may be maintained from about 0.5% w/w to about 1.5% w/w after removal of the solvent. The moisture content of the reaction mass may be maintained, if required by adding a quantity of water. The moisture content may be maintained for about 5 minutes to about 2 hours, for example from about 10 minutes to about 1 hour before isolating the pure levofloxacin hemihydrate. Time more than this may result in the formation of the monohydrate.

In one general aspect the solution containing the crude levofloxacin may be treated with charcoal before removing the solvent. The charcoal treatment may be carried out under heating conditions or it may be carried out at a lower temperature.

In another general aspect the slurry containing the product may be cooled prior to isolation to obtain better yields of the levofloxacin hemihydrate and the product may be washed with a suitable solvent.

In another general aspect there is provided a method of treating a patient in need of antimicrobial therapy using therapeutically effective amount of the pure levofloxacin hemihydrate.

The details of one or more embodiments of the inventions are set forth in the description below. Other features, objects and advantages of the inventions will be apparent from the description and claims.

### DESCRIPTION OF THE DRAWINGS

FIG. 1 is X-ray powder diffraction pattern of pure levofloxacin hemihydrate prepared as described herein.

### DETAILED DESCRIPTION OF THE INVENTION

The inventors have developed an efficient process for the preparation of pure levofloxacin hemihydrate, by obtaining a solution of crude levofloxacin in one or more organic solvents; removing the solvent; maintaining a moisture content of reaction mass from about 0.5% w/w to about 1.5% w/w; and isolating the pure levofloxacin hemihydrate. The inventors also have developed pharmaceutical compositions that contain the pure form of the levofloxacin hemihydrate, in admixture with one or more solid or liquid pharmaceutical diluents, carriers, and/or excipients. These pharmaceutical compositions may be used for treating a patient in need of antimicrobial therapy.

In general, the solution of crude levofloxacin may be obtained by dissolving crude levofloxacin in a suitable solvent. Alternatively, such a solution may be obtained directly from a reaction in which levofloxacin is formed. The solvent containing crude levofloxacin may be heated to obtain a solution. It can be heated from about 30° C. to about reflux temperature of the solvent used, for example from about 30° C. to about 100° C. In particular, it can be heated from about 40° C. to about 60° C. It can be heated from about 15 minutes to about 10 hours. More particularly, it can be heated for about 2-3 hours.

The solvent may be removed from the solution by a technique which includes, for example, distillation, and distillation under vacuum.

The term “suitable solvent” includes any solvent or solvent mixture in which crude levofloxacin can be solubilized, including, for example, chlorinated hydrocarbons, hydrocarbons, esters, water, and mixtures thereof. Examples of chlorinated hydrocarbons include solvents such as chloroform, dichloromethane, and 1,2-dichloroethane. A suitable hydrocarbon includes one or more of hexanes, cyclohexane, and toluene. Examples of esters include solvents such as methyl acetate, ethyl acetate, and isopropyl acetate. Mixtures of all of these solvents are also contemplated.

In another aspect, a base may be added prior to heating the solvent containing crude levofloxacin. Any base may be used, for example triethylamine.

The moisture content of the reaction mass can be maintained from about 0.5% w/w to about 1.5% w/w by adding a quantity of water. The moisture content can be maintained for about 5 minutes to about 2 hours, for example from about 10 minutes to about 1 hour before isolating the pure levofloxacin hemihydrate. Time more than this is not required as it results in the formation of the monohydrate.

The product obtained may be further or additionally dried to achieve the desired moisture values. For example, the product may be further or additionally dried in a tray drier, dried under vacuum and/or in a Fluid Bed Drier.

The pure levofloxacin hemihydrate has a purity of more than 99%. More particularly, the purity of levofloxacin hemihydrate is more than 99.5%.

The pure levofloxacin hemihydrate thus obtained is essentially free of monohydrate. The term “essentially free” of monohydrate refers to levofloxacin hemihydrate in which levofloxacin monohydrate is not detectable by X-ray diffraction technique. The limit of detection of levofloxacin monohydrate in levofloxacin hemihydrate by X-ray diffraction technique being 0.25%.

Methods known in the art may be used with the process of this invention to enhance any aspect of this invention. For example, the solution containing the crude levofloxacin may be treated with charcoal before removing the solvent. The charcoal treatment may be carried out under heating conditions or it may be carried out at a lower temperature. The slurry containing the product may be cooled prior to isolation to obtain better yields of the levofloxacin hemihydrate and the product may be washed with a suitable solvent.

The present invention is further illustrated by the following example which is provided merely to be exemplary of the inventions and is not intended to limit the scope of the invention. Certain modifications and equivalents will be apparent to those skilled in the art and are intended to be included within the scope of the present invention.

### Example

Preparation of Levofloxacin Hemihydrate

Levofloxacin crude (1.25 Kg) was taken in dichloromethane (25 Lit) at ambient temperature, followed by the addition of ethyl acetate (18.75 Lit). It was stirred and triethylamine (0.525 Lit) and water (0.75 Lit) were added. The reaction mixture was heated to reflux (50-52° C.) for about 2 hours. It was cooled to 30-35° C. and treated with activated charcoal. It was further heated to reflux temperature for 30 minutes and filtered hot through a hyflo bed. The hyflo bed was washed with dichloromethane (5.0 Lit). The combined filtrate was collected and solvent was removed. Water (350 ml) was added and it was stirred for 10 minutes. The resulting slurry was cooled to 35° C. and solid was filtered. The solid was washed with ethyl acetate and was dried. This results in levofloxacin hemihydrate having purity more than 99.5% by HPLC. The physical data of the pure levofloxacin hemihydrate are as follows:

Moisture content (Karl-Fischer's method): 2.4%

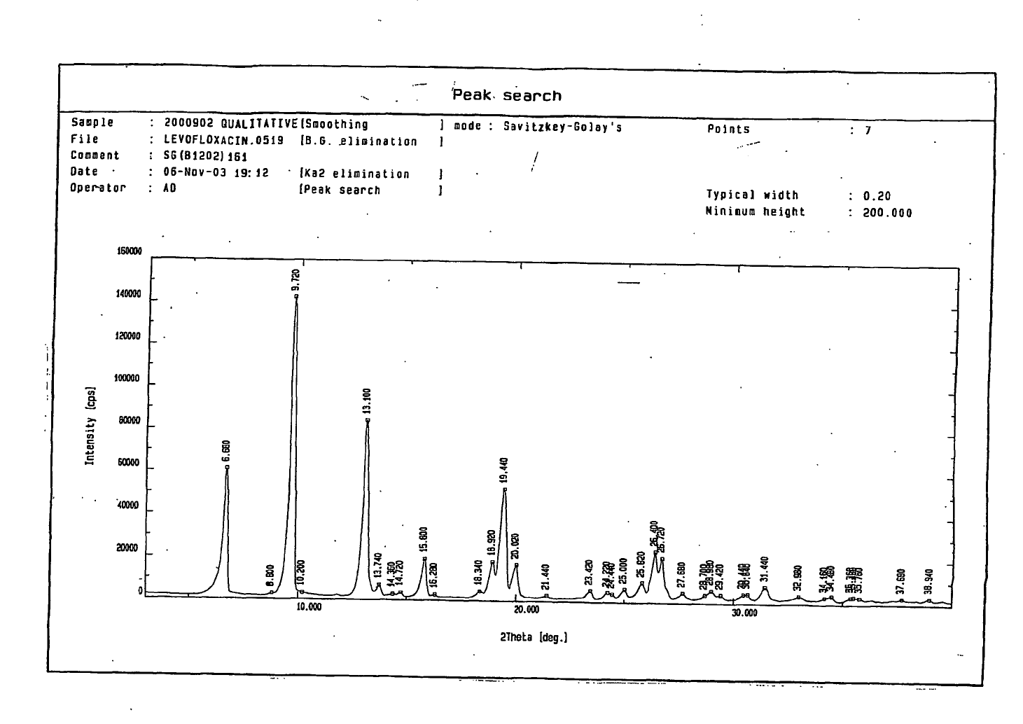

Powder X-ray diffraction determined by Rigaku D MAX 2500 is shown in FIG. 1.

While the present invention has been described in terms of its specific embodiments, certain modifications and equivalents will be apparent to those skilled in the art and are intended to be included within the scope of the present invention.

1. A process for the preparation of pure (S)-9-fluoro-3-methyl-10-(4-methyl-1-piperazinyl)-7-oxo-2,3-dihydro-7H-pyrido[1,2,3-di][1,4]-benzoxazine-6-carbxoylic acid hemihydrate (levofloxacin hemihydrate) of Formula I, 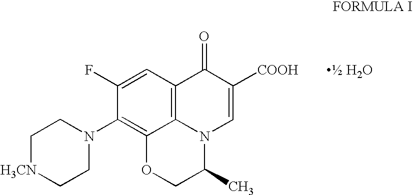
the process comprising obtaining a solution of crude levofloxacin in one or more organic solvents; removing the solvent; maintaining a moisture content of reaction mass from about 0.5% w/w to about 1.5% w/w; and isolating the pure levofloxacin hemihydrate.

2. The process of claim 1, wherein the solution of crude levofloxacin is obtained by heating the solvent.

3. The process of claim 2, wherein the heating temperature ranges from about 30° C. to about 100° C.

4. The process of claim 3, wherein the heating temperature ranges from about 40° C. to about 60° C.

5. The process of claim 1, wherein the organic solvent comprises one or more of chlorinated hydrocarbon, hydrocarbon, ester, or mixtures thereof.

6. The process of claim 5, wherein the chlorinated hydrocarbon comprises one or more of chloroform, dichloromethane, and 1,2-dichloroethane.

7. The process of claim 6, wherein the chlorinated hydrocarbon is dichloromethane.

8. The process of claim 5, wherein the hydrocarbon comprises one or more of hexane, cyclohexanes, and toluene.

9. The process of claim 5, wherein the ester comprises one or more of methyl acetate, and ethyl acetate.

10. The process of claim 9, wherein the ester is ethyl acetate.

11. The process of claim 1, wherein removing the solvent comprises one or more of distillation, and distillation under vacuum.

12. The process of claim 1, further comprising adding a base before removal of the organic solvent.

13. The process of claim 11, wherein the base is triethylamine.

14. The process of claim 1, wherein the moisture content of the reaction mass is maintained by adding water.

15. The process of claim 1, wherein isolating the pure levofloxacin hemihydrate comprises one or more of filtration, filtration under vacuum, decantation, and centrifugation.

16. The process of claim 1, further comprising additional drying of the product obtained.

17. The process of claim 1, further comprising forming the product obtained into a finished dosage form.

18. The A method of treating a patient of claim 25 wherein the levofloxacin hemihydrate is prepared by the process of claim 1.

19. Levofloxacin hemihydrate having a purity of more than 99.0% by HPLC.

20. The levofloxacin hemihydrate of claim 19, having a purity of more than 99.5% by HPLC.

21. The levofloxacin hemihydrate of claim 19, having a purity of more than 99.8% by HPLC.

22. Pure levofloxacin hemihydrate, which is essentially free of levofloxacin monohydrate.

23. The pure levofloxacin hemihydrate of claim 21, wherein the levofloxacin hemihydrate has the X-ray diffraction pattern of FIG. 1.

24. A pharmaceutical composition comprising a therapeutically effective amount of pure levofloxacin hemihydrate; and one or more pharmaceutically acceptable carriers, excipients or diluents.

25. A method of treating a patient in need of an antimicrobial therapy, the method comprising providing a dosage form to said patient that includes pure levofloxacin hemihydrate.
